(19) United States
(12) Patent Application Publication
(10) Pub. No.: US 2011/0230907 A1
(43) Pub. Date: Sep. 22, 2011

(54) ARTERIAL TAMPONADE DEVICE AND METHOD

(75) Inventors: Patrick J. Fitzgerald, Poway, CA (US); R. Todd McKinney, Rancho Santa Fe, CA (US); John A. Simpson, Carlsbad, CA (US)

(73) Assignee: SINOCCLUSIVE LLC, Rancho Santa Fe, CA (US)

(21) Appl. No.: 13/076,811

(22) Filed: Mar. 31, 2011

Related U.S. Application Data
(63) Continuation-in-part of application No. 12/751,731, filed on Mar. 31, 2010.
(60) Provisional application No. 61/315,761, filed on Mar. 19, 2010.
(60) Provisional application No. 61/315,766, filed on Mar. 19, 2010.

(51) Int. Cl. A61M 29/00

(52) U.S. Cl. 606/199; 606/191

(57) ABSTRACT

An arterial tamponade device is expandable between a collapsed condition and an expanded position and has opposite ends, with an enlarged pad at one or both ends. The device is inserted into a body cavity in its collapsed condition and is released at a predetermined location so that opposite ends of the device are biased away from one another and apply pressure to opposite wall regions of the body cavity before the device is fully expanded, with a pad at one end positioned to apply pressure to a predetermined tissue area which includes a blood vessel so as to occlude or partially occlude the vessel and reduce or cut off blood flow to the body cavity. The pad may have a bulbous projection configured to engage a depression of a body cavity wall region in which a blood vessel is located.

### RELATED APPLICATION

The present application is a Continuation-In-Part of patent application No. 12/751,731 filed on Mar. 31, 2010, which claims the benefit of co-pending U.S. provisional patent application No. 61/315,761 filed on Mar. 19, 2010 and provisional patent application No. 61/315,766 filed on Mar. 19, 2010, the contents of each of which are incorporated herein by reference in their entirety.

### BACKGROUND

1. Field of the Invention

The present invention relates generally to tamponade devices used to block or restrict blood flow, and is particularly concerned with an intranasal tamponade device to reduce nasal bleeding by applying force to a blood vessel to partially or completely occlude blood flow.

2. Related Art

Bleeding during a surgical procedure is a problem for the surgeon because it limits visibility and can add to overall procedural time. This is particularly true in endonasal surgeries, both due to the fact that the nasal cavity is already narrow and dark, and due to the fact that the nasal cavity contains many small blood vessels that bleed easily, obscuring the surgical field. Currently, medication is used to reduce bleeding during surgery, or applied to the nose after surgery. Post-operative bleeding is also often controlled by nasal packing and packing devices such as gauze. Removal of such packing is typically quite painful and uncomfortable for the patient.

Because the nose has many small blood vessels fed from the sphenopalatine artery, cauterizing or severing of this artery is sometimes used for treating chronic nose bleeds.

There is therefore a need for a mechanism to improve visualization during surgery, particularly endonasal surgery, and to reduce bleeding more effectively without cauterization of arteries.

### SUMMARY

Embodiments described herein provide for a tamponade device to reduce bleeding during and after surgery or to reduce spontaneous nasal bleeding as a result of other nasal facial trauma.

According to one embodiment, a tamponade device is provided which is designed to be inserted into a body cavity in a compressed or collapsed state and which is configured to be expanded after insertion so that opposite ends of the device engage and apply pressure to opposite areas of the body cavity. At least one end of the device comprises a foot or pad which is positioned to apply pressure to an area of the body cavity which includes an artery or other blood vessel, so as to occlude the vessel and reduce or cut off blood flow through the artery or blood vessel. This can be useful during surgery to reduce bleeding in the surgical field, after surgery to reduce post-operative bleeding, or to reduce bleeding as a result of trauma or various medical conditions.

The tamponade device in one embodiment comprises a relatively thin elongate member or strut of resilient or shape memory material with at least one enlarged foot or pressure pad secured to one end. Both the rod and the foot are of suitable biocompatible materials appropriate for use in the body. The elongate member may be a wire, rod, or flat strip or ribbon of resilient plastic, metal or a balloon mechanism or the like. The enlarged foot or pad may be generally disc-shaped or oval, with an outwardly facing, tissue engaging surface, or may be of other shapes such as polygonal or irregular shapes, and the tissue engaging surface may be concave or convex. In one embodiment, the foot or pad has an outer face having a central, arcuate or convex bulbous protrusion designed to engage in an anatomical depression found in the sphenopalatine artery (SPA) foramen of the nasal space and to apply pressure through the mucosal tissue to tamponade the underlying SPA.

The foot or pad may alternatively comprise a bent end portion of the elongate member. In one embodiment, an enlarged foot or pad is provided at both ends of the elongate member, and the opposite feet engage opposing regions of the body cavity. Alternatively, the opposite end of the elongate member may directly contact the opposing area of the body cavity to hold and support the device. The dimensions and material of the elongate member are such that the feet or pads can be deformed inwardly towards one another with the elongate member compressed into a U or V-like shape for insertion into a body cavity, for example using a medical grasping tool, a tubular sheath, an endoscope or a customized delivery system. When in the proper position, the tool or sheath can be withdrawn or the device can be pushed out of the sheath so that the elongate member springs out, forcing the feet apart into engagement with opposing regions of body tissue in the body cavity. The device is positioned so that the enlarged foot or pressure pad at the one end of the device engages a key point or area of the body that includes an artery or other blood vessel, applying pressure to occlude the blood vessel and reduce or cut off blood supply to the body region involved. The opposite end or foot engages an opposite area of the tissue so as to hold the device in place.

In one embodiment, the elongate member may be made from a shape memory metal or plastic material suitable for use in the body, such as nitinol (nickel and titanium alloy) or the like. The foot or feet may be of a suitable biocompatible material such as hydroxyapetite or a suitable biocompatible plastic or other material which may be injection molded around the end of the bent wire or ribbon.

The device can help to cut down bleeding during or after surgery, bleeding as a result of injury, or as result of certain medical conditions, by occluding an artery which provides blood supply to the area involved. In one embodiment the tamponade device is designed as an intranasal arterial tamponade device with the pressure pad on one leg positioned to occlude the sphenopalatine artery of the nose. The sphenopalatine artery (nasopalatine artery) passes through the sphenopalatine foramen into the cavity of the nose, at the back part of the superior meatus. The device can be positioned low in the nose out of the typical sinonasal surgical field, using a nasal endoscope device which may have position markings relative to the artery occluding foot to aid in proper positioning in the nasal cavity. The feet or pressure pads may be designed with a non-slip outer surface texture, for example with a roughened surface or a surface with plural dimples or bumps, to assist in holding the device in place, in addition to the spring pressure applied by the legs. Once placed in position, the non-slip texture of the feet, combined with the spring force applied by the flexible legs of the device, supports the feet in position while occluding or at least partially occluding the underlying artery.

In one embodiment, the feet can be drug-eluting so as to slowly release a drug over time, for example a drug which further reduces bleeding or which combats inflammation, infection and/or pain. For example, the feet in one embodiment have a coating of material which holds and elutes a drug into the tissue which they engage, similar to known drug-eluting stents.

According to another aspect, a method of temporarily applying pressure to an area of a body cavity wall including a blood vessel and occluding or substantially occluding the vessel to stop or reduce blood flow is provided, in which an expandable tamponade device is held in a compressed, unexpanded condition and inserted into a body cavity with a pressure pad at one end of the device oriented to face towards a predetermined region of the cavity wall which includes a blood vessel. The compressed tamponade device is released at a predetermined position in the body cavity so that it expands towards a fully expanded condition and opposite ends of the device engage and press against opposing wall regions of the body cavity before the device is fully expanded, with the pressure pad pressing against the predetermined region of the cavity wall including the blood vessel and applying sufficient pressure to at least partially occlude the blood vessel and reduce blood flow to the body cavity. The device may be held in the compressed condition by a suitable insertion tool or a nasal endoscope or insertion sheath until it reaches the desired position in the body cavity.

The embodiment of the device designed for intranasal use may also include a spring loaded turbinate extension from the elongate member which is secured at one end to the elongate member and is biased outwardly away from the elongate member in the relaxed condition of the device. The turbinate extension extends in a direction towards the footed end of the device, and is collapsed against the elongate member during insertion of the device. When the device is released from its compressed condition, the turbinate extension springs out away from the elongate member. In another embodiment, the turbinate extension may be of a malleable material that is physically bent into the desired position after insertion of the device. The turbinate extension is configured to retract or push the middle turbinate away from a surgical site when the enlarged foot is properly located so as to apply pressure to the sphenopalatine artery. This improves intraoperative visualization and can also be used to stent the middle meatus open in the postoperative period.

The arterial tamponade device described above is particularly useful as an intraoperative device to reduce bleeding in the operative field, particularly for endoscopic endonasal procedures which generally produce significant bleeding, so as to provide improved visualization of the surgical site. The device may also be used to reduce bleeding as a result of trauma or medical conditions such as nose bleed. One known treatment for nose bleeds, as an alternative to lengthy periods of nasal packing, is permanent vascular ligation or invasive arterial embolization which permanently blocks blood flow. Instead of permanent closing of an artery, the tamponade device could be installed to block blood flow temporarily, and then removed once bleeding is under control.

Other features and advantages of the present invention will become more readily apparent to those of ordinary skill in the art after reviewing the following detailed description and accompanying drawings.

### BRIEF DESCRIPTION OF THE DRAWINGS

The details of the present invention, both as to its structure and operation, may be gleaned in part by study of the accompanying drawings, in which like reference numerals refer to like parts, and in which:

FIG. 1 is a front elevation view of a first embodiment of an arterial tamponade device in a relaxed, expanded condition;

FIG. 2 is a front elevation view of a modified tamponade device in a relaxed, expanded condition;

FIG. 3 is a front elevation view of the device of FIG. 1 or FIG. 2 in a partially compressed condition;

FIG. 4 is a side elevation view of the device in the compressed condition of FIG. 3;

FIG. 5 is a coronal cross-sectional view through a nasal cavity illustrating placement of the tamponade device of FIGS. 1 to 4 to occlude the sphenopalatine artery;

FIG. 6 is an enlarged view of a first foot portion of the device of FIG. 3 engaging the area of the lateral nasal wall including the sphenopalatine artery;

FIG. 7A is a side elevation view illustrating the device of FIGS. 1 to 6 confined in an introducer sheath or nasal endoscope for introduction and placement in the nasal cavity;

FIG. 7B is a view similar to FIG. 7A but illustrating the device partially pushed out of the sheath and expanding towards opposing areas of the nasal wall;

FIG. 7C is a view similar to FIGS. 7A and 7B but illustrating the device as it is pushed completely out of the sheath into an expanded condition pushing against opposing areas of the nasal wall;

FIG. 8 is a front elevation view of a modified intranasal arterial tamponade device in a partially compressed condition similar to FIG. 3;

FIG. 9 is a sagittal cross-sectional view through a nasal cavity illustrating placement of the modified tamponade device of FIG. 8 in the same position as illustrated in FIG. 5 for the embodiment of FIGS. 1 and 2;

FIG. 10 is a front elevation view of another modified tamponade device in a fully relaxed, expanded condition;

FIG. 11 is a front elevation view of intranasal arterial tamponade device of FIG. 10 in an introducer sheath prior to placement in a nasal cavity;

FIG. 12 is a coronal cross-sectional view through a nasal cavity illustrating placement of the tamponade device of FIGS. 10 and 11 to occlude the sphenopalatine artery and retract the middle turbinate out of a surgical field;

FIG. 13 is a rear perspective view of another embodiment of an arterial tamponade device in a relaxed, expanded condition;

FIG. 14 is a front perspective view of the arterial tamponade device of FIG. 13;

FIG. 15 is an enlarged view of the pressure pad for applying pressure to tamponade the sphenopalatine artery (SPA);

FIG. 16A is a perspective view of one embodiment of the strut or pad connecting member of the device of FIGS. 13 to 15;

FIG. 16B is a perspective view of a modified strut or connecting member of smaller dimensions than FIG. 16A;

FIG. 17 is a longitudinal cross-section of the arterial tamponade device on the lines 17-17 of FIG. 14;

FIG. 17A is a longitudinal cross-section similar to FIG. 17 illustrating an alternative, angled orientation for the pressure pad;

FIG. 18 is an enlarged cross sectional view on the lines 18-18 of FIG. 14;

FIG. 19 is a coronal cross-sectional view through a nasal cavity similar to that of FIG. 5, but illustrating placement of the tamponade device of FIGS. 13 to 18 to occlude the sphenopalatine artery;

FIG. 20 is an enlarged view of the foot plate or pressure pad of the device of FIGS. 13 to 18 with the bulbous protrusion engaging the hollow depression of the sphenopalatine artery foramen;

FIG. 21A is a side elevation view illustrating another embodiment of an insertion device for placement of the tamponade device of any of the preceding embodiments in a body cavity;

FIG. 21B illustrates a modification of the insertion device of FIG. 21A;

FIG. 22 is a side elevation view illustrating another alternative insertion tool or device in a retracted position;

FIG. 23 illustrates the device of FIG. 22 with the inner shaft in an extended position for placement or retrieval of a tamponade device;

FIG. 23A is an enlarged view of the hook at the end of the inner shaft of FIGS. 22 and 23; and

FIG. 23B is a top plan, enlarged view of the hook at the end of the inner shaft.

### DETAILED DESCRIPTION

Certain embodiments as disclosed herein provide for a tamponade device configured for placement between opposing areas of a body cavity so as to apply pressure against a predetermined area of tissue, such as a wall of a cavity in the body, including a blood vessel, so as to temporarily occlude the blood vessel and stop or reduce blood flow through the vessel. In one embodiment, the device is designed for placement in a nasal cavity to occlude the sphenopalatine artery supplying blood to the nose.

After reading this description it will become apparent to one skilled in the art how to implement the invention in various alternative embodiments and alternative applications. However, although various embodiments of the present invention will be described herein, it is understood that these embodiments are presented by way of example only, and not limitation.

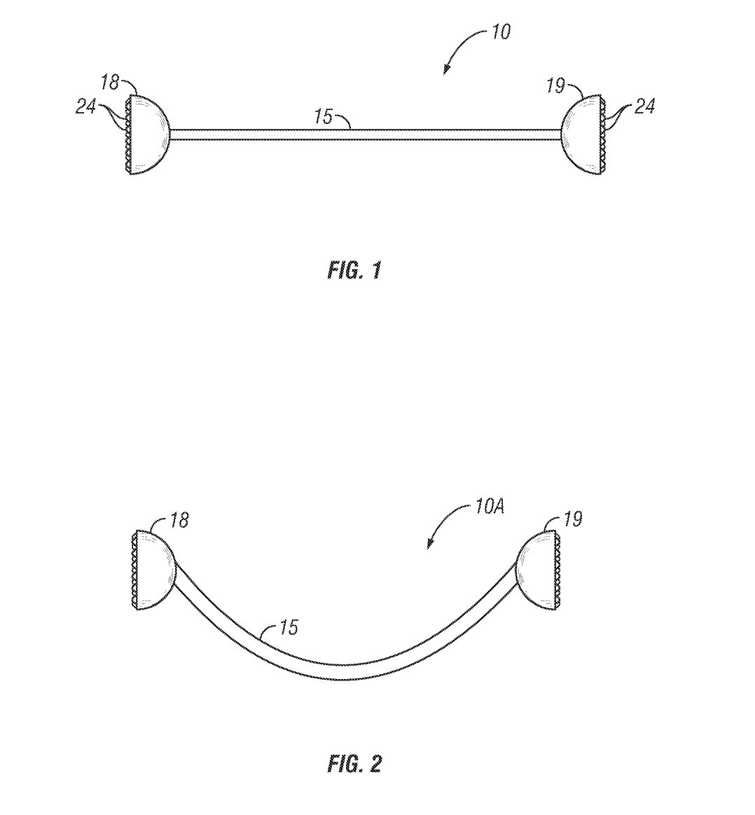

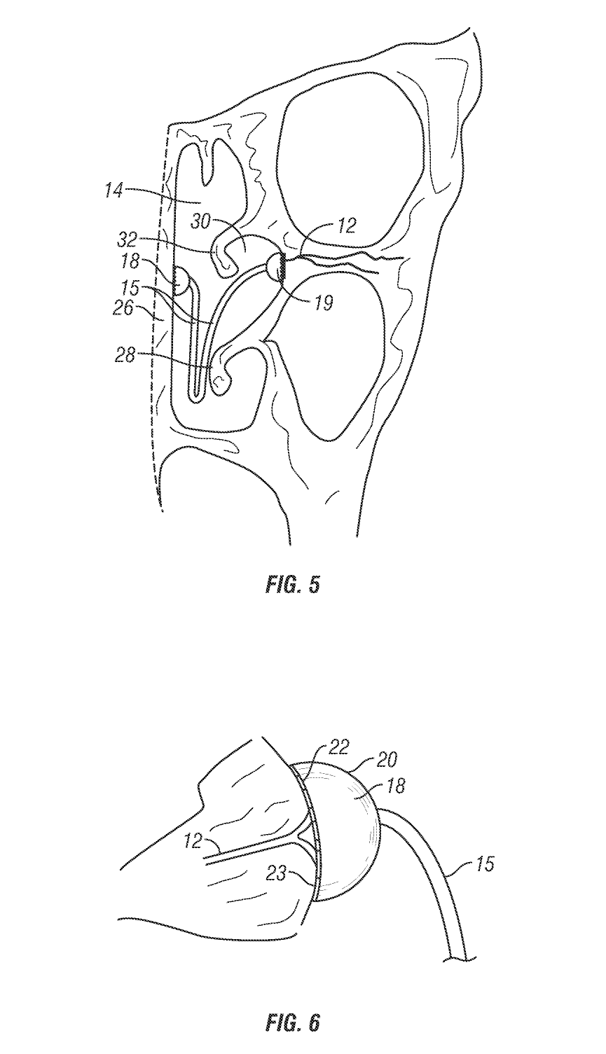

FIGS. 1, 3 and 4 illustrate a first embodiment of a tamponade device 10 which is designed to temporarily occlude a blood vessel such as an artery or vein, while FIGS. 5 and 6 illustrate the device in use. FIG. 2 illustrates a modified tamponade device 10A which is of slightly different configuration from device 10. In the illustrated embodiment, the device 10 or 10A is configured for placement in a nasal cavity 14 and is designed for temporarily occluding the sphenopalatine artery (SPA) 12 of the nose. In alternative embodiments, the device may be of appropriate shape and dimensions for placement in other parts of the body or body cavities to apply pressure against blood vessels in tissue, such as cavity walls, so as to temporarily occlude the vessel or at least reduce blood flow through the vessel and reduce bleeding as a result of surgery, trauma or the like, as discussed in more detail below.

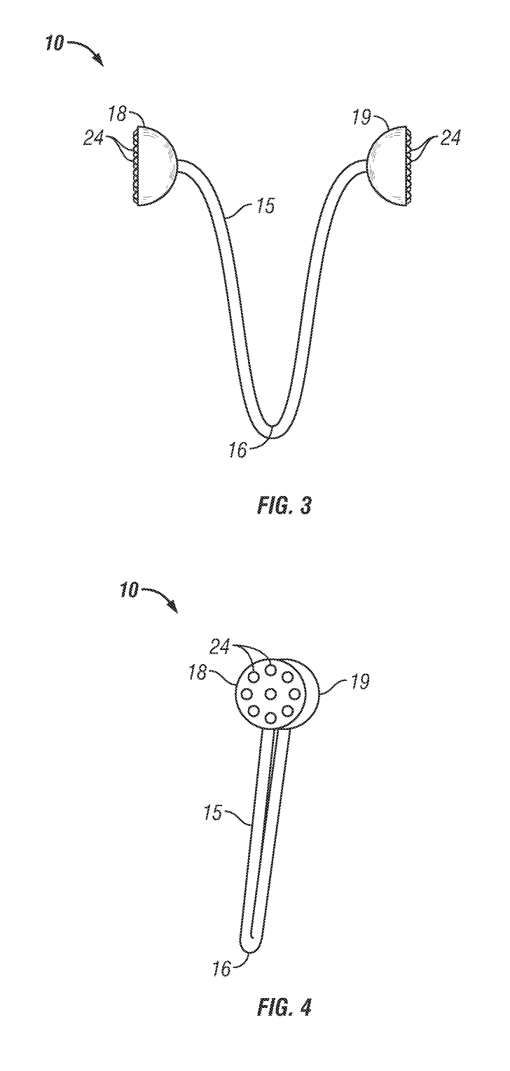

FIG. 1 illustrates device 10 in a relaxed, fully expanded condition, while FIGS. 3 and 4 illustrate the device in a partially compressed or distorted condition. Device 10 basically comprises a relatively thin elongate member or strut 15 of resilient or shape-memory material with an enlarged foot or pressure applying pad 18, 19 secured to each end of member 15. Member 15 may be formed from a length of a suitable springy or resilient metal or plastic wire, rod, or flat ribbon or strip of a material which is biocompatible with body tissues, for example a shape memory alloy material such as nitinol or the like. In the embodiment of FIG. 1, the elongate member 15 is straight in the fully expanded, relaxed condition, while the modified embodiment of FIG. 2 is slightly curved when fully expanded. Other shapes may be used, such as a wide V-shape, U shape or the like. The elongate member may be solid or tubular.

In the illustrated embodiment, the feet or pads 18, 19 are substantially identical in shape and dimensions and each foot is of disc-like, round or oval shape with a rounded inner surface 20 and a cupped or concave outer surface 22 configured to engage an opposing surface of a body cavity, as illustrated in the enlarged sectional view of FIG. 6. In alternative embodiments, one foot may be larger than the other foot, and the feet may be of different shapes, as described in more detail below. The device may be compressed between the expanded, relaxed condition as illustrated in FIG. 1 and a compressed or deformed condition as illustrated in FIGS. 3, 4 and 7A, for example by pushing the feet or pads 18, 19 or opposite regions of member 15 towards one another, forming a generally U-like or looped shape with a bend 16 as in FIG. 3, for example.

The feet may be of any suitable biocompatible material such as hydroxyapatite or a biocompatible injection molded plastic or other material, and may be rigid or substantially rigid, and either solid or hollow. The shape of the outer, tissue engaging surface 22 is designed to substantially match the shape of the area of a body cavity which it is intended to engage, for close mating engagement and pressing against the area. Other shapes may be used for this surface, depending on the body cavity area to be engaged, such as convex, concave, substantially flat, or the like. In alternative embodiments, the material of feet 18, 19 may be slightly deformable to better match the shape of an opposing surface against which they are pressed on installation.

The body or tissue engaging surfaces 22 of the feet may also be designed as non-slip or slip-resistant surfaces. The surfaces may be roughened or may have a series of small protrusions 24 as indicated in FIGS. 1 to 4 so as to help grip and hold the device in place once positioned in the body cavity. In an alternative embodiment, the tissue engaging surfaces 22 may have indentions or dimples, or may have small openings or pores. The inner surfaces 20 are secured to the ends of the respective legs by adhesive or the like, or the feet may be injection molded over ends of the elongate member 15, which may be bent or hooked at its ends to better attach the feet. In one embodiment, the feet may be of metal and may be suitably welded to the ends of the legs. A twist or swivel joint (not illustrated) may be provided between the feet and legs to help in appropriate positioning of the outer, body engaging surfaces 22. The feet may also be an uninterrupted extension of the metal legs of the device.

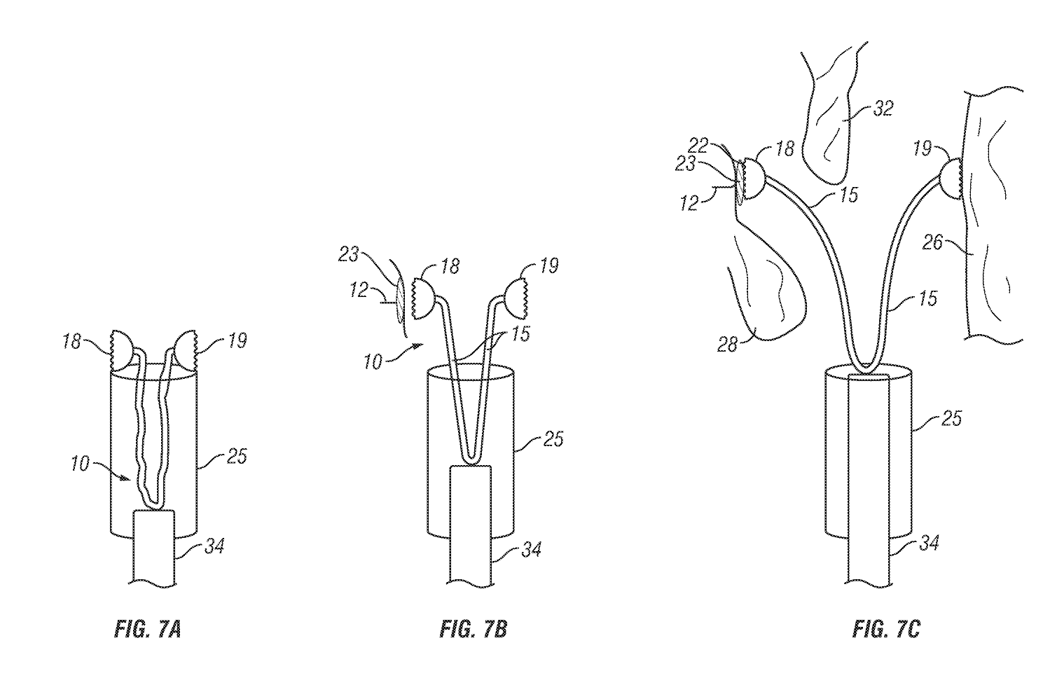

Device 10 may be positioned in a body cavity by means of a standard surgical grasping instrument, via an introducer such as a cylindrical sheath or endoscope 25, as illustrated in FIGS. 7A to 7C and described in more detail below, or via other customized delivery systems. Device 10 is designed to be collapsed from its original, unstressed or fully expanded shape as illustrated in FIG. 1 into a compact, compressed configuration as illustrated in FIG. 7A for installation purposes, so that it can be moved to a selected region in a body cavity without significantly contacting structures in the path to the desired region. The legs spring out towards the extended, unstressed position of FIG. 1 when released from the grasping instrument or introducer, and engage opposite wall regions of the body cavity while the device is still partially compressed, so that the partially compressed elongate member 15 applies a biasing force pressing the feet or pads 18, 19 against the opposite wall regions. In the embodiment illustrated in FIGS. 1 to 4, device 10 is designed for intranasal placement in a nasal cavity as illustrated in FIG. 5, with the spring force in the compressed elongate member or ribbon 15 urging the opposing feet 18, 19 against predetermined opposing areas of the nasal cavity. The device is positioned so that foot 18 is urged against predetermined area 23 of the lateral nasal wall including the sphenopalatine artery 12 and foot 19 bears against an opposite area of the nasal septum 26, with one of the legs extending over the inferior turbinate 28 and foot 18 positioned in the middle meatus area 30 between the inferior turbinate 28 and middle turbinate 32.

The foot 18 which is designed to engage the tissue surface including the sphenopalatine artery (or a blood vessel in a different body cavity in alternative embodiments) is suitably configured to apply sufficient pressure over an area of the artery to at least substantially occlude blood flow in the artery, and also to minimize trauma to the tissue. In the illustrated embodiment, this foot is circular or disc shaped with an arcuate tissue engaging surface. The tissue contacting surface may be convex or concave. The foot may be solid or hollow, and the tissue engaging surface may have an open center or core. Other peripheral shapes may be used for the feet, such as circular, oval, rectangular, polygonal, or irregular shapes, and the opposing inner and outer surfaces of the foot may be of the same or different shapes, and may be substantially flat, convex, concave, or the like. In other alternatives, the foot 18 may be a V-shaped or two pronged, fork-like wire extension at the end of the elongate member. The second foot is designed to assist in holding the device in position and may be of the same or different shape from foot 18.

The distance between the sphenopalatine artery (SPA) and the septum in typical individuals is approximately 1.2-1.8 cm, with an average of 1.5 cm. The variability is on the septum side and to a lesser extent the skull size of the patient. The dimensions of device 10 in the fully expanded, relaxed condition of FIG. 1 are such that, when positioned and released at the desired location in nasal cavity 14 as in FIG. 3, the device cannot fully expand and the member 15 is still partially compressed or deformed inwardly from its original straight or partially curved shape so as to apply spring force against the opposing cavity surfaces via feet 18 and 19. The device may be of varying lengths. In one embodiment, the fully expanded spacing between the tissue engaging surfaces at the ends or feet of the device is of the order of 20 to 30 mm, and in one embodiment the fully expanded spacing is around 25 mm. The feet or pressure pads 18, 19 may be circular or oblong in shape and of the order of around 5 to 12 mm in cross-sectional dimension or diameter and 1 to 1.5 mm in thickness. In one embodiment, both pads have a diameter of around 7.5 mm. The artery side foot 18 and septum side foot 19 may be of different sizes in alternative embodiments. In one embodiment, the artery side foot was round with a diameter of around 7.5 mm while the septum side foot was oblong with a maximum dimension of around 10 mm. In one embodiment for use as an SPA tamponade, device 10 is designed so that foot 18 applies pressure of around 150 gm per sq. cm (around 2 psi) or more to the opposing tissue surface of the lateral nasal wall. In some cases, the device may be designed to apply a significantly higher amount of pressure. Devices of different sizes may be provided for different size nasal cavities or for use in different body cavities. The device is designed to be of relatively low profile when installed in the selected position in the nose.

When placed as illustrated in FIG. 5, the disc-like feet or pressure pads 18, 19 engage and apply pressure against the nasal mucosa and bone. The device is of relatively low profile when installed and is placed low in the nose out of the normal surgical field (see FIG. 9). Foot 18 is configured to exert pressure on the underlying sphenopalatine artery 12 so as to temporarily occlude the artery and cut off blood flow to the nasal cavity. This artery is a major blood supply to the nose and occluding it temporarily, for example during endoscopic sinonasal surgeries, reduces intra-operative bleeding and, more importantly, improve visualization and reduce surgical procedure times during such procedures. At the conclusion of surgery, the device 10 can be removed and discarded, or may be left in place for a time period after surgery if needed.

FIGS. 7A to 7C illustrate one embodiment of a delivery system for placing the arterial tamponade device 10 of FIGS. 1 to 4 in the nasal cavity at the desired location as illustrated in FIGS. 5 and 6. As illustrated in FIG. 7A, the bent connecting strut 15 of device 10 is initially positioned within sheath 25 in a compressed, deformed state, with feet 18, 19 outside the sheath 25 and opposing portions of strut 15 pushed close together and forming a loop or U-like shape, and the sheath is then inserted to the desired location within the nasal cavity. Markings on the introducer sheath may indicate the orientation to be used so as to correctly position one of the feet 18 to engage the key area 23 of the lateral nasal wall that includes the sphenopalatine artery 12. Once the sheath is at the desired position and at the correct orientation for positioning the feet, pusher 34 is used to push the device 10 out of the sheath and into position, as illustrated in FIGS. 7A and 7B. As the device exits the sheath, the opposite portions of the elongate member spring apart and the feet 18, 19 are pushed against opposing regions of the nasal cavity wall, specifically against the area 23 of the lateral nasal wall in the middle meatus which includes the sphenopalatine artery and against the opposing region of the nasal septum 26. The device is designed so that the pressure applied by the device frame or legs against area 23 is sufficient to hold the device in place and to occlude or at least substantially occlude blood flow from the sphenopalatine artery. The roughened consistency of the outer surfaces of the feet 18, 19 helps to hold the feet against slipping once they engage the walls.

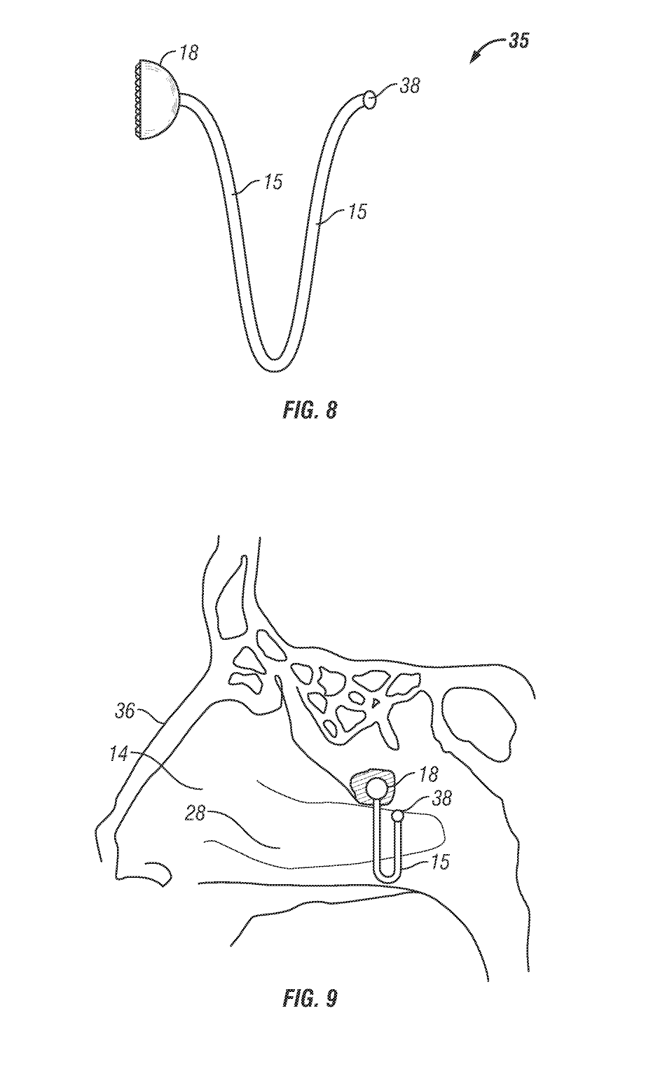

FIGS. 8 and 9 illustrate a modified tamponade device 35, with FIG. 8 illustrating the device in the same, partially compressed position as device 10 in FIG. 3, with the sectional view taken from a different direction. FIG. 9 is a side sectional view of the nose 36 and one nasal cavity 14. This shows how the device is positioned low in the nose and away from most sino-nasal surgery sites. Some parts of the device 35 are identical to the previous embodiment, and like reference numbers are used for like parts as appropriate. The only difference is that one of the feet 19 of the previous embodiment is removed and replaced with a smaller foot 38 designed simply to contact the opposing structures of the nose to provide the desired fixation and support of the device. Alternatively, the foot 19 or 38 could be removed altogether and the frame leg 15 could directly contact the opposing nose structure or nasal septum, or could be bent into a loop or similar shape at the end for contacting the septum. In other alternative embodiments, foot 19 may be larger than foot 18 or the feet may be of different shapes.

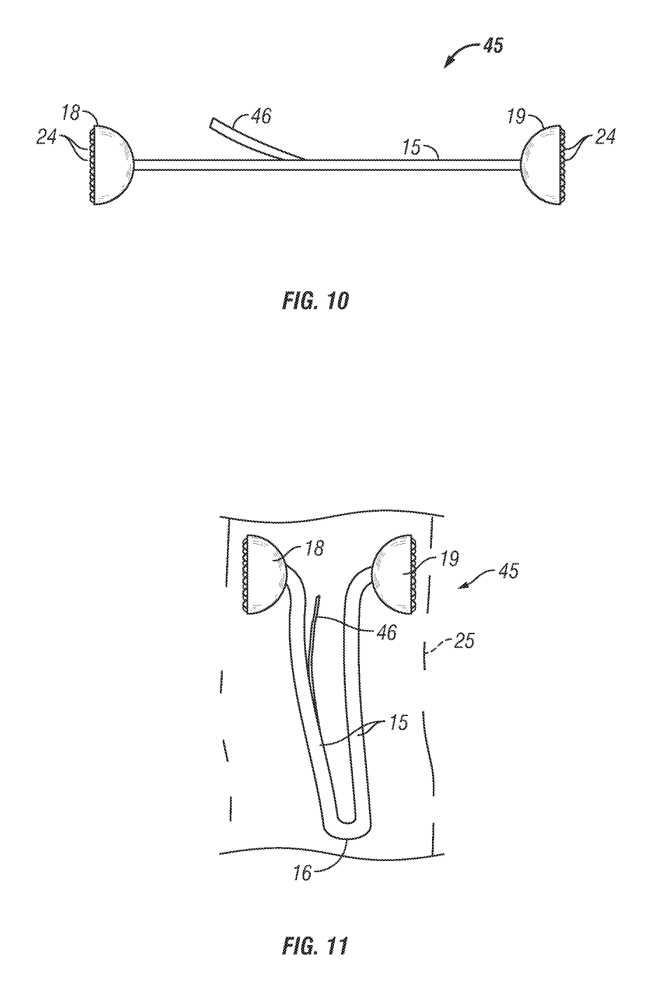

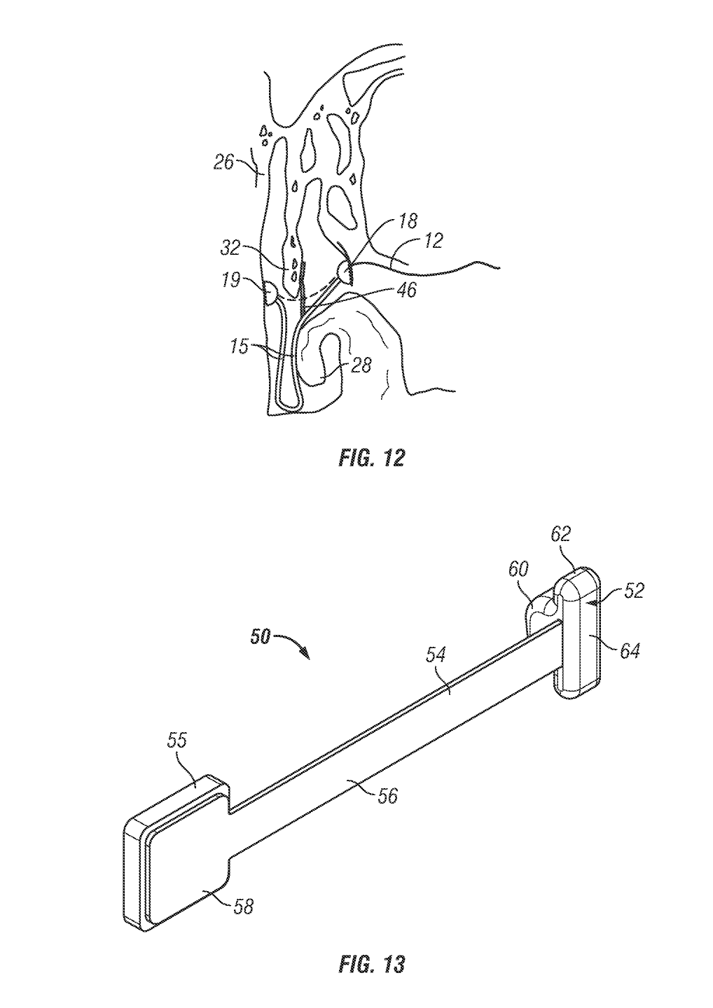

FIGS. 10 and 11 illustrate an arterial tamponade device 45 according to another embodiment, while FIG. 12 illustrates the device 45 positioned in the nasal cavity. This embodiment is similar to the embodiment of FIGS. 1 to 4, and like reference numbers are used for like parts as appropriate. However, device 45 has a turbinate extension 46 of the same thickness and material as elongate member 15. Extension 46 is secured to one face of the elongate member 15 and extends generally towards foot or pressure pad 18. Extension 46 is shaped so that it bends away from member 15 in the fully expanded, unstressed condition of FIG. 10. FIG. 11 illustrates the device in a collapsed, compressed state inside sheath 25, similar to the position for device 10 in FIG. 7A, in which opposite portions of member 15 are urged inwardly about bend 16 with turbinate extension 46 on the inner side of the resultant V or U shape. When the device 45 is released from the sheath 25 at the desired location in the nasal cavity, the compressed opposite portions of elongate member 15 on opposite sides of bend 16 spring outwards until the feet 18, 19 engage opposing wall regions of the nasal cavity 14, as in the previous embodiments, with foot 18 engaging area 23 including the sphenopalatine artery and foot 19 engaging an opposing area of the nasal septum 26. At the same time, extension 46 springs away from member 15, engaging and retracting the middle turbinate 32, as illustrated in FIG. 12. The turbinate extension 46 allows for turbinate medialization during surgery and in the postoperative period if desired. The extension 46 improves intraoperative visualization and stents the middle meatus open in the postoperative period. In an alternative embodiment, turbinate extension 46 may be of a malleable material that is physically bent into the desired position after insertion of the device.

In the above embodiments, the foot or feet may also contain a suitable medication and may slowly release the medication during and after surgery. For example, the tissue engaging surfaces of the feet may have a drug-eluting coating of a material which elutes a drug onto the adjacent tissue, such as a sinus treatment drug, an infection or inflammation combating drug, or a drug which reduces bleeding, or an anesthetic. The medication is designed to be gradually released from the feet onto the adjacent cavity wall surfaces over time.

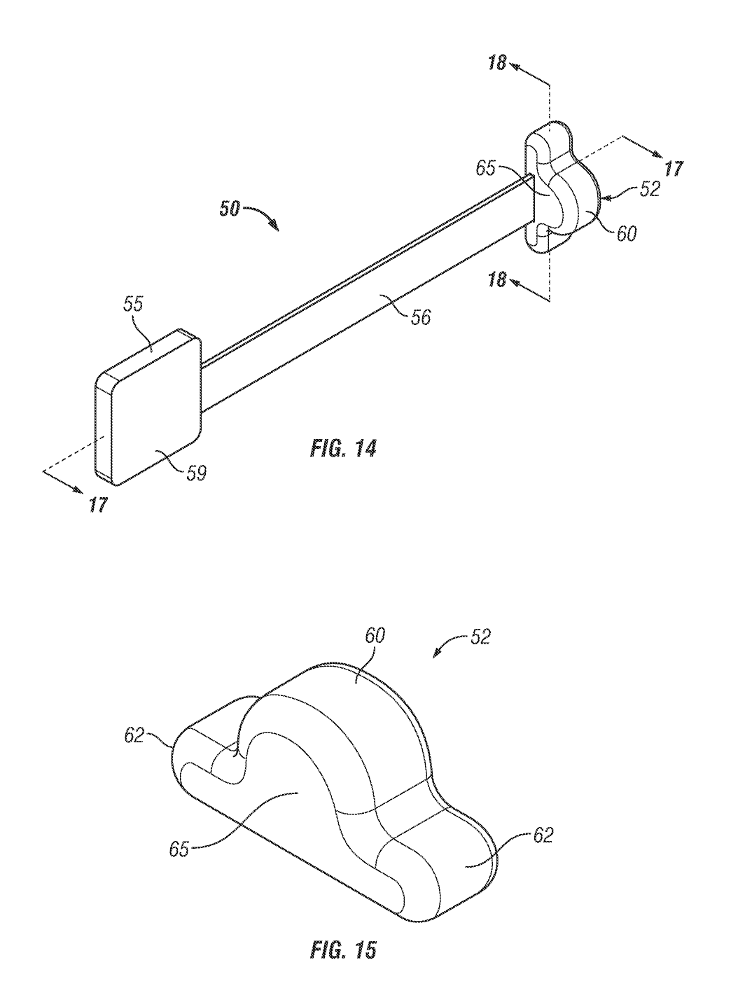

FIGS. 13 to 18 illustrate a second embodiment of an arterial tamponade device 50 which is designed to temporarily occlude a blood vessel such as an artery or vein, for example the sphenopalatine artery (SPA) of the nose, while FIGS. 19 and 20 illustrate the device in use. In the illustrated embodiment, the device 50 is configured for placement in a nasal cavity 14 and is designed for temporarily occluding the sphenopalatine artery 12 of the nose. In alternative embodiments, the device may be of appropriate shape and dimensions for placement in other parts of the body or body cavities to apply pressure against blood vessels in tissue, such as cavity walls, so as to temporarily occlude the vessel or at least reduce blood flow through the vessel and reduce bleeding as a result of surgery, trauma or the like, as discussed in more detail below.

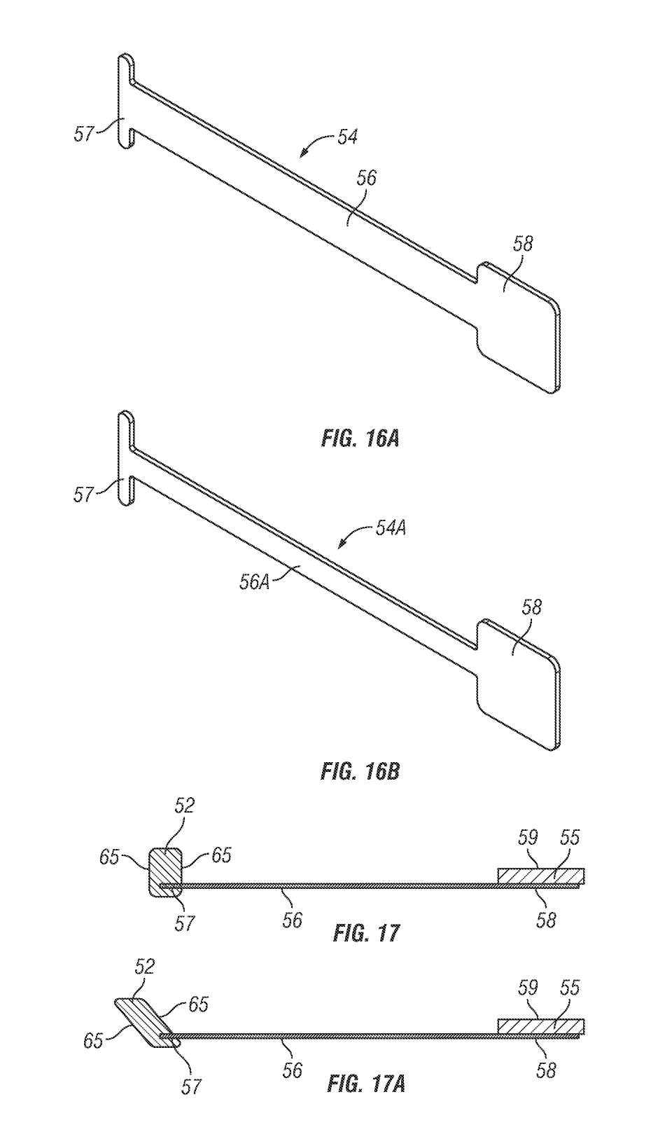

Device 50 basically comprises a relatively thin elongate connecting member or strut 54 of resilient or shape-memory material with a specially shaped foot or pressure applying pad 52 secured at one end of member 54 and a rectangular or square pad 55 secured at the opposite end. In use, pad 52 is adapted to apply pressure to the sphenopalatine artery or SPA, while pad 55 is designed to bear against an opposite region of the nasal septum 26, as illustrated in FIGS. 19 and 20. FIGS. 13, 14 and 17 illustrate device 50 in a relaxed, fully expanded condition in which strut 54 is straight, while FIG. 15 illustrates the SPA foot or pad 52 and FIG. 16 illustrates the elongate connecting member or strut 54 which connects the pad 52 to the opposite end foot or pad 55 which is adapted to bear against the nasal septum 26, as explained in more detail below.

Member or connecting strut 54 is formed from a length of a suitable springy or resilient metal or plastic flat ribbon or strip of a material which is biocompatible with body tissues, for example a shape memory alloy material such as nitinol or the like. The connecting strut 54 in FIGS. 13 to 14, 16 and 17 is a flat strip, but may be rod-shaped or tubular in alternative embodiments. As illustrated in FIG. 16A, strut 54 has a connecting portion 56 extending between a T-shaped end 57 and an enlarged, square base plate 58 at the opposite end. As illustrated in FIG. 17, the T-shaped end 57 is embedded in the SPA pad 52 and the thin, nasal septum engaging pad 55 is bonded to the base plate 58 at the opposite end of strut 54.

As illustrated, strut 54 has opposite, flat faces and the pads 52 and 55 each have a tissue engaging face which faces in the same direction as a first one of the flat faces, such that the pads face outward when the strut is bent into a generally U-shaped or V-shaped configuration with the second face of the strut facing inward. Member or strut 54 may be made in different lengths and widths to accommodate different nose dimensions. FIG. 16B illustrates one alternative in which the connecting portion 56A is about half the width of connecting portion 56 of FIG. 16A. In one embodiment, the width of connecting portion 56 is around 0.13 inches while the width of connecting portion 56A is around 0.08 inches. The length of connecting portion 56 between the ends 57 and 58 is in the range from 1.2 to 1.4 inches, and in one embodiment strut 54 was provided with connecting portions of lengths 1.24 and 1.34 inches, respectively, with the longer and shorter connecting portions both being provided in the alternative 0.13 and 0.08 inch widths. The thickness of strut in one example is around 0.015 inches, making it relatively flexible or bendable into a compressed condition similar to that illustrated for device 10 of the first embodiment in FIGS. 3, 4 and 7.

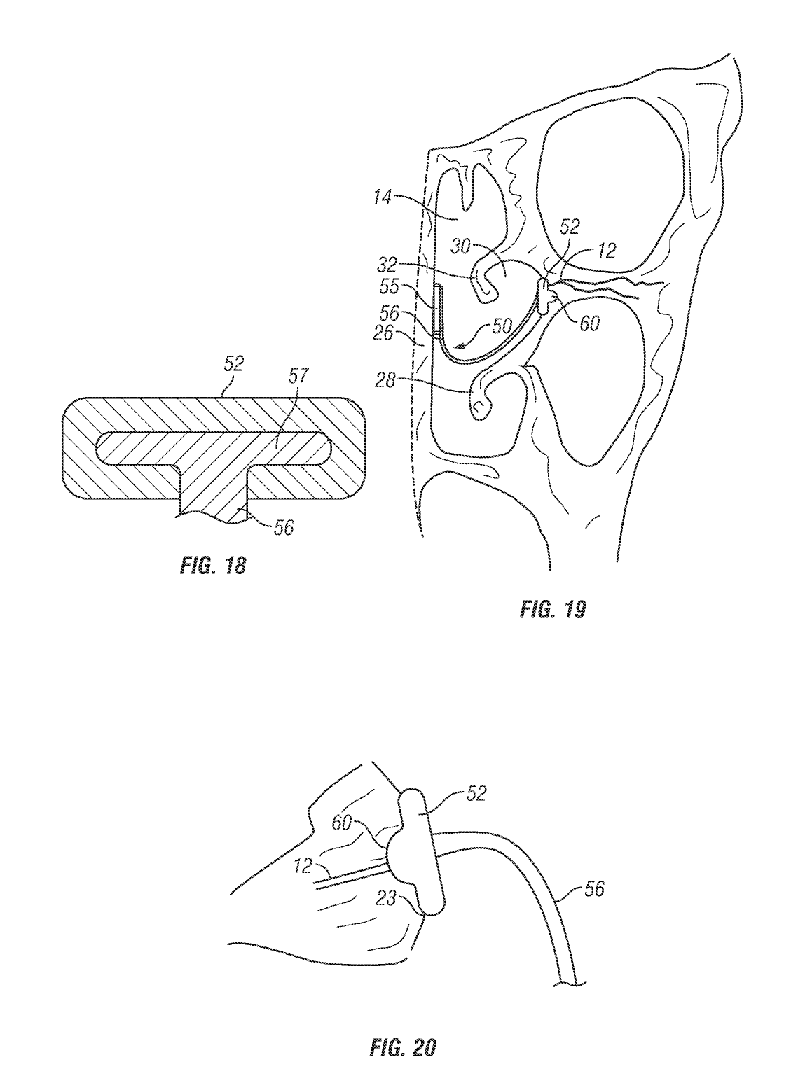

Septum engaging or septal pad 55 is a relatively thin, square pad of suitable resilient material such as polyester felt, and has a substantially flat outer or tissue engaging face 59. SPA pad 52 is of any suitable biocompatible material such as hydroxyapatite or a biocompatible injection molded plastic or other material, and may be rigid or substantially rigid. In one embodiment, pad 52 is of Nylon 12 with a biocompatible blue colorant. The pad may also be made of other plastics materials such as polyetheretherketone (PEEK™ or TECAPEEK™). The pad is formed with a small, bulbous protrusion 60 in its outer, SPA or tissue engaging face, and is molded around the T-shaped end 57 of the elongate connecting member or strut 54, as best illustrated in FIGS. 17 and 18. As illustrated in FIG. 15, pad 52 is molded with rounded ends 62 and a flat inner face 64. In one embodiment, the T-shaped end 57 of strut 54 is centered in a mold cavity for forming pad 52, and the molten material is then injected into the cavity to surround T-shaped end 57 so that it is embedded in the formed pad.

In one example, the height of the rounded end portions on each side of protrusion 60 is of the order of 0.08 inches, while the height of the rounded or bulbous protrusion is of the order of 0.18 inches. The length between opposite ends 62 is of the order of 0.36 inches while the length of protrusion 60 is of the order of 0.18 inches or half the length of the pad. The width of the pad between opposite side faces 65 is of the order of 0.12 inches. The width of square pad 55 is around 0.32 inches in one embodiment. Thus, each pad has a width or length in a direction traverse to connecting portion 56 which is at least twice the width of portion 56. Different dimensions may be used in alternative embodiments.

The SPA pad is shaped so that the bulbous protrusion 60 engages the artery at the point where the artery exits the lateral wall of the nasal space. This area, known as the SPA foramen, consists of a hollow depression behind a bony crest along the posterior lateral wall of the nasal anatomy that “houses” the sphenopalatine artery (SPA) as it exits the skull and enters the nasal tissues. The device 50 is positioned in the nose in the same way as described above for the first embodiment, using an insertion tool or delivery system such as tool or system 25 of FIGS. 7A to 7C to hold the device in a compressed, generally U-shaped condition with the septal and SPA engaging faces of pads 55 and 52, respectively, facing outward. The shape of the SPA pad or foot 52, with its bulbous protrusion 60, is designed to seek out the depression found in the SPA foramen as the device is released from the delivery system. The tension exerted through the strut of the released device allows the SPA foot or pad 52 to lodge itself behind the bony crest with the protrusion 60 centered in the depression of the foramen so as to apply pressure through the mucosal tissue and tamponade the underlying SPA, as generally illustrated in FIGS. 19 and 20. When positioned in nasal cavity 14 as illustrated, the spring force in the compressed or bent elongate member or strut 54 urges the opposing feet 52, 55 against predetermined opposing areas of the nasal cavity. The device is positioned so that foot 52 is urged against predetermined area of the lateral nasal wall including the sphenopalatine artery 12, with protrusion 60 engaging the depression in the SPA foramen, while foot or septal pad 55 bears against an opposite area of the nasal septum 26, with strut 54 extending over the inferior turbinate 28 and pad 52 positioned in the middle meatus area 30 between the inferior turbinate 28 and middle turbinate 32.

In FIGS. 13, 14 and 17, the SPA engaging foot 52 is oriented perpendicular to the connecting strut 54. In the modified embodiment of FIG. 17A, the foot or pad 52, or at least the bulbous protrusion which bears against the artery in the nose, is angled outward at an angle of 20 to 40 degrees to the perpendicular direction of FIG. 17. This alternative may be used to accommodate different nasal anatomies.

As in the previous embodiments, the foot or feet 52 and 54 of tamponade device 50 may also contain a suitable medication which is slowly released during and after surgery. For example, the tissue engaging surfaces of the feet may have a drug-eluting coating of a material which elutes a drug onto the adjacent tissue, such as a sinus treatment drug, an infection or inflammation combating drug, or a drug which reduces bleeding, or an anesthetic. The medication is designed to be gradually released from the feet onto the adjacent cavity wall surfaces over time.

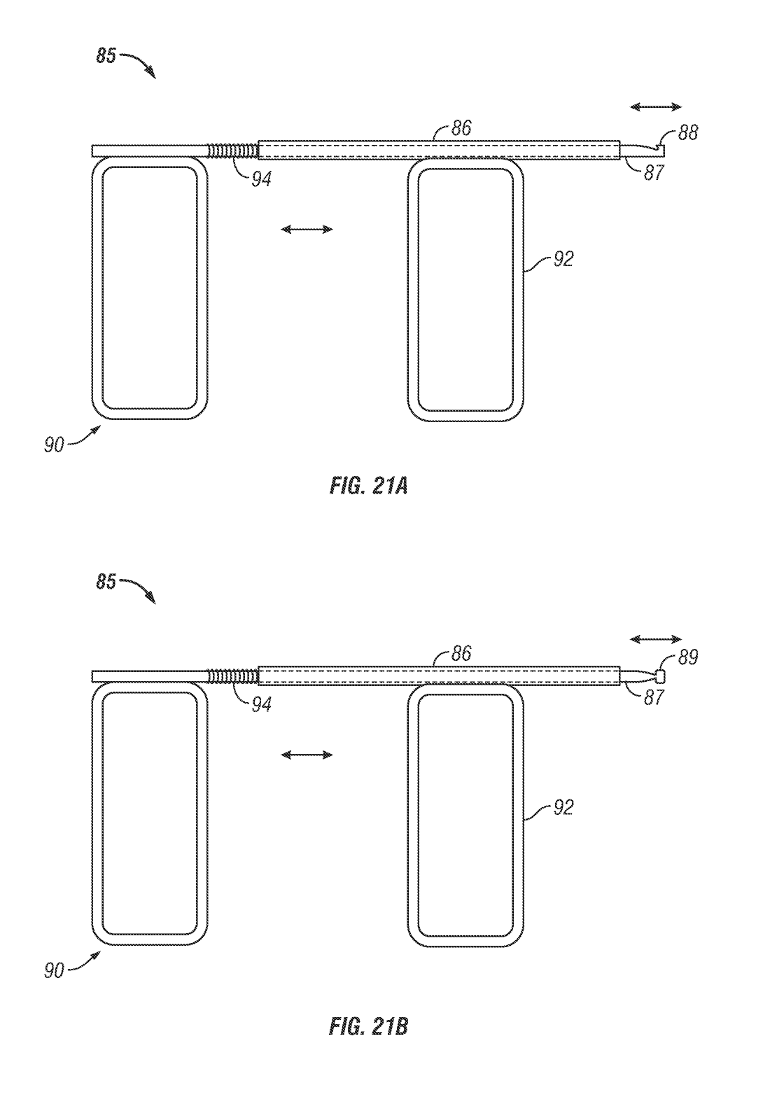

FIG. 21A illustrates another embodiment of an introducer or insertion tool 85 for the arterial tamponade device of any of the above embodiments. Insertion tool 85 is similar to insertion device 25 of FIG. 7A to 7C and comprises a tubular sheath or endoscope 86 in which the device 10 is retained (as in FIG. 7A) as it is inserted into the nasal cavity or other body cavity. In this embodiment, pusher member 87 has a hook 88 at its end which engages over the looped end of the compressed tamponade device when in the position shown in FIG. 7A. FIG. 21B illustrates a modification of the tool of FIG. 21A with an alternative hooked end 89. All other parts are identical to those of FIG. 21A and have been given like reference numerals. A first actuator or handle loop 90 is coupled to the pusher 87. A second actuator or handle loop 92 is coupled to the sheath 86. Movement of the first actuator 90 (rotational or longitudinal movement) relative to second actuator 92 causes movement of the pusher relative to the sheath. A spring 94 or other source of compressive force or resistance can be used to provide a selected amount of resistance to moving the first actuator so as to move the end of the pusher 87 with the hook within a certain range of and beyond the end of the sheath. The resistance can decrease the likelihood of accidentally deploying the tamponade, and also biases the hooked end of the pusher into the shaft when the handle loops are released. The tubular sheath 86 may be of round or square tubing, and the pusher member has a shaft of corresponding round or square cross-section for sliding engagement in sheath 86.

The hook or retainer 88 reduces the risk of the device springing out of the sheath too quickly and engaging the wrong positions on the nasal cavity walls. Once the device is properly positioned, the pusher member is advanced to disengage the hook 88 from the elongate wire member, then the pusher is rotated to clear the wire before being retracted back into the sheath. An additional embodiment of the introducer sheath and pusher member may be in a square or rectangular shape to maximize collapsed curvature and thus minimize permanent set of the elongate member of the tamponade device.

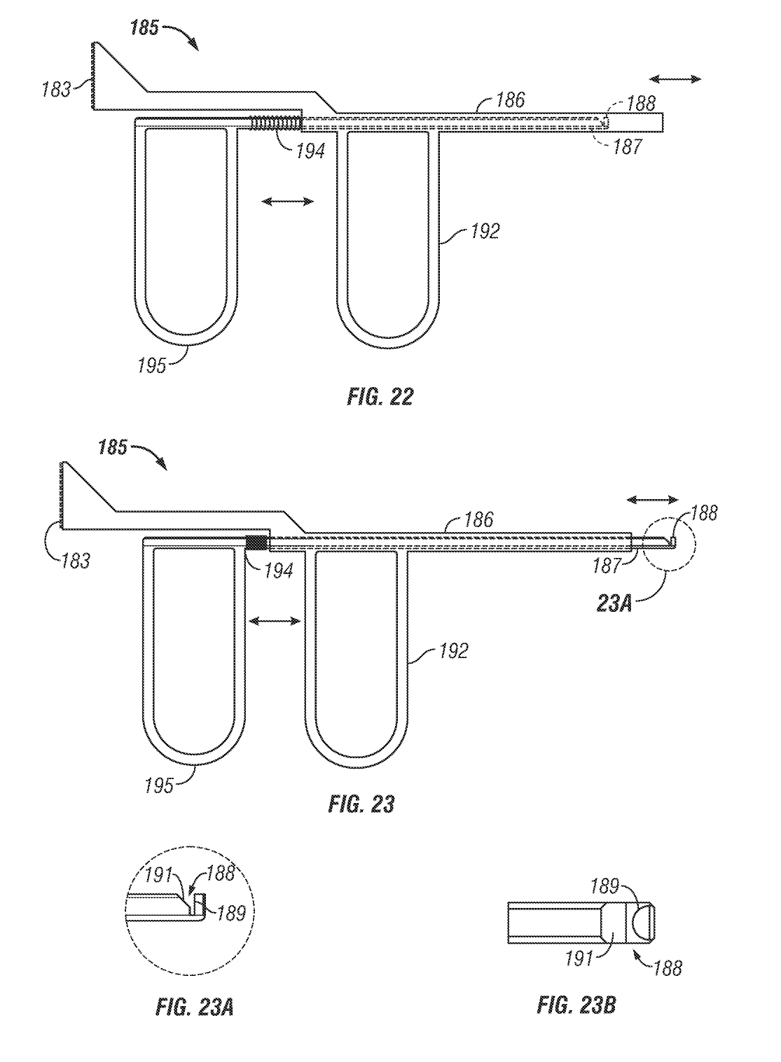

FIGS. 22 to 23B illustrate another embodiment of an introducer or insertion tool 185 for the arterial tamponade device of any of the above embodiments. Insertion tool 185 is similar to tool or device 85 and comprises a tubular sheath or endoscope 186 in which at least the connecting strut of the tamponade device is retained (as in FIG. 7A) as it is inserted into the nasal cavity or other body cavity. A first handle loop 192 extends from a distal end portion of sheath 186. In this embodiment, a pusher member or inner shaft 187 has distal end portion engaging in tubular sheath 186 and a proximal end portion extending rearwards out of the proximal end of the sheath. Member 187 has a hook 188 at its distal end which can be engaged over the looped end of the tamponade device when in the extended position shown in FIG. 23 in which the hook projects out of the distal end of the sheath, and holds the tamponade device in a compressed condition within the end of the tubular sheath when in the retracted position of FIG. 22. As in the previous embodiment, the tubular portion of sheath 186 and the slidably engaged pusher member are of corresponding round or square cross-section, or of other mating cross-sectional shapes. The tubular sheath 186 has rearwardly extending portion offset from the central axis of the tubular portion and spaced from the proximal end portion of the pusher member, with a tailfin or pusher 183 at its rear or proximal end. The proximal end portion of the pusher member 187 has a second handle loop 195 which extends from pusher member 187 in the same direction as first handle loop 192. Compression spring 194 extends between the second handle loop 195 and the proximal end of the tubular sheath 186. As best illustrated in FIGS. 23A and 23B, the pusher member 187 has a hook 188 at its forward end which has a radiused end face 189 to retain the bent end of the connecting strut of the tamponade device prior to insertion without kinking, and a bevel or tapered surface 191 facing end face 189 which enables the hook to engage or disengage the tamponade device from a larger range of approach angles, and directs the bent end of the device into the narrower, inner end portion of the hook.

The delivery system or insertion tool 185 is assembled in the same fashion as the insertion tool 85 of FIGS. 21A and 21B, with forward end portion of the inner shaft 187 extending into the tubular portion of the outer sheath or endoscope and the compression spring located between the two handle loops in order to maintain a biasing force which urges the inner pusher member or shaft 187 into the retracted position of FIG. 22. When a tamponade device is hooked onto the hook 188 at the end of pusher member 187, this biasing force tends to pull the hooked-and-folded device deeper into the hollow sheath 186 unless being intentionally deployed by the user. In this embodiment, the handles are now formed integrally with the hollow member and hook-bearing shaft member, respectively, rather than being brazed or welded on as in the embodiment of FIGS. 21A and 21B.

To deploy the hooked and folded tamponade device, the user squeezes both handle loops 192 and 195 toward one another, causing the distal or hooked end of the hook-bearing inner shaft to advance out of the hollow member, as illustrated in FIG. 23, and thereby push out the constrained device. This action is essentially the same as the previous design. The fully radiused island feature or inner end 189 of the hook reduces the risk of the tamponade device kinking at this contact point, and the adjacent bevel 191 enables the hook to engage or disengage the device from a wider range of approach angles than if no bevel were present.

To remove a tamponade device from the nose, or pick up a tamponade device for later placement in the nasal cavity, the user pushes with his/her thumb on the ridged surface of the proximal “tailfin” 183 while pulling back on the proximal handle loop, causing the distal end of the hollow member or sheath 186 to advance over the exposed hook-bearing distal end of the inner shaft 187 and thereby fold and retract a properly-hooked tamponade device. The addition of the tailfin to the hollow member enables retrieval to be routinely performed with one hand, while the user holds an endoscope with the other. Lacking this tailfin, the previous embodiment requires both handle loops to be spread apart to fold and withdraw the device, an action which typically required two hands.

In each of the above embodiments, the elongate member or strut extending between the feet or end pads of the tamponade device is of bendable, resilient metal or plastics material which is biocompatible. When compressed into a generally U-shape and released, the resilient elongate member seeks to return to the original, relaxed and non-compressed state with sufficient tensile strength to counteract mucosal and arterial wall pressure as well as arterial hydrostatic pressure. In other words, the foot or SPA pad which engages the area of the nasal wall including the sphenopalatine artery is pressed against the mucosa and underlying arterial wall with sufficient force to occlude or at least substantially occlude the artery. The device uses the bony boundaries of the nasal wall to provide the counter force necessary for stability and pressure. As noted above, the device may be removed and discarded after use. It may be installed prior to surgery in order to reduce blood in the operative field which can interfere with visualization, and may be left in place for a time period after surgery to reduce post-operative bleeding. The device is of low profile so that it is out of the normal surgical field and does not interfere with the surgeon's view of the surgical site. A safety string or tether (not illustrated) may be attached to the device and extend out of the nose for assistance in locating and removing the device after surgery or after sufficient healing has occurred. In some embodiments, the entire tamponade device may be of bioabsorbable or bioresorbable material so that removal after surgery or treatment is not needed.

In addition to use during surgery for improved visualization purposes, the above devices may also be used for other purposes. For example, the tamponade device may be left in place after surgery for reduction of post-operative bleeding and also as a post-operative stenting device. The device may also be used as an alternative to lengthy periods of nasal packing, invasive arterial embolization, or permanent vascular ligation procedures for treating chronic nose bleeds or refractory epitaxis.

Although the above embodiments describe use of an arterial tamponade device in the nasal cavity, a similar device of appropriately modified dimensions may be used in other regions of the body to temporarily reduce or eliminate blood flow through an artery if needed during surgery, following surgery or other trauma, or due to a medical condition which causes excessive bleeding. The dimensions of the device may be adjusted as appropriate so that the feet or pads 18, 19 or 52, 55 bear against opposing regions of a selected body cavity with one foot pressing against a part of the cavity wall including an artery or other blood vessel so as to occlude or at least substantially occlude the vessel and reduce or cut off blood flow.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly limited by nothing other than the appended claims.

1. A tamponade device for applying pressure against a predetermined area of a wall or cavity in the body including a blood vessel, comprising:
an elongate strut of resilient, biocompatible material having opposite first and second ends;
a first pad of biocompatible material secured to the first end of the strut and having a tissue engaging face configured to engage a predetermined area of a body cavity including a blood vessel;
a second pad of biocompatible material secured to the second end of the strut and having a tissue engaging face configured to engage an opposing portion of a body cavity; and
the strut being configured for bending into a generally U-shaped, compressed condition with the tissue engaging faces of the pads facing outward, the compressed strut being configured to bias the pads outwards towards an expanded condition when released.

2. The device of claim 1, wherein the elongate strut comprises a length of biocompatible, resilient material.

3. The device of claim 2, wherein the biocompatible material is nitinol.

4. The device of claim 1, wherein the strut is substantially straight in a fully expanded, non-bent condition.

5. The device of claim 1, wherein the device is arcuate in the fully expanded, relaxed condition.

6. The device of claim 1, wherein the device is of predetermined dimensions for placement in a nasal cavity in the bent, generally U-shaped condition and the first pad has tissue engaging surface configured to engage a predetermined area of a lateral nasal wall including the sphenopalatine artery (SPA).

7. The device of claim 6, wherein the tissue engaging surface of the first pad has a bulbous protrusion configured to engage a depression in the SPA foramen which houses the SPA.

8. The device of claim 7, wherein the second pad is a generally rectangular pad of compressible material having an outer, flat face comprising the tissue engaging face.

9. The device of claim 7, wherein the first end of the strut is embedded in the first pad.

10. The device of claim 9, wherein the first end of the strut is T-shaped.

11. The device of claim 7, wherein the strut is a flat member having opposite side edges and opposite first and second flat faces, and the pads are secured to opposite ends of the strut with the tissue engaging faces of the pads facing in the same direction as the first flat face of the strut, whereby the tissue engaging faces of the pads face outwards when the strut is bent into a generally U-shaped condition with the second flat face of the strut facing inward.

12. The device of claim 7, wherein the first pad is of injection molded plastic material.

13. The device of claim 12, wherein the plastic material is nylon.

14. The device of claim 1, wherein the tissue engaging faces of the pads have a roughened, slip resistant surface texture.

15. The device of claim 1, wherein the pads are of different sizes.

16. The device of claim 1, wherein the pads are of different shapes.

17. The device of claim 16, wherein the tissue engaging face of the first pad has a bulbous protrusion and the tissue engaging face of the second pad is substantially flat.

18. The device of claim 1, wherein the length of the strut between the first and second pads is in the range from around 1.2 to 1.4 inches.

19. The device of claim 18, wherein the strut is a flat strip of flexible material and a portion of the strut extending between the first and second pads has a transverse width in the range from 0.08 to 0.14 inches.

20. The device of claim 1, wherein the strut is a flat strip having a portion between the pads which has a first width in a transverse direction parallel to a flat face of the strip, and the transverse width of each pad in a direction parallel to the transverse direction is at least twice the first width.

21. The device of claim 17, wherein the bulbous protrusion extends perpendicular to the strut.

22. The device of claim 17, wherein the bulbous protrusion extends at an angle to the strut in a direction away from the second pad.

23. The device of claim 17, wherein the angle of the bulbous protrusion is between 20 and 40 degrees to a direction perpendicular to the strut.

24. A method of temporarily applying pressure to an area of a body cavity wall including a blood vessel and occluding or substantially occluding the vessel to stop or reduce blood flow, comprising:
bending an elongate, resilient strut of a tamponade device from a substantially straight, relaxed condition into a bent condition in which opposite ends of the device are moved towards one another into a generally U-shaped, compressed configuration with tissue engaging faces of first and second pads located at opposite ends of the strut facing outwards and away from one another;
holding the device in an insertion device while in the bent, generally U-shaped configuration;
inserting the bent, generally U-shaped tamponade device into a body cavity with the first pad at one end of the bent strut oriented to face towards a predetermined region of the cavity wall which includes a blood vessel; and
releasing the tamponade device from the insertion device at a predetermined location in the body cavity, whereby the strut expands outwards from the generally U-shaped configuration towards a fully expanded condition and the pads at opposite ends of the strut engage and press against opposing wall regions of the body cavity before the strut is fully expanded, the first pad configured to press against the predetermined region of the cavity wall including the blood vessel and to apply sufficient pressure to at least partially occlude the blood vessel and reduce blood flow to the body cavity.

25. The method of claim 24, wherein the body cavity is a nasal cavity and the first pad is pressed against a predetermined region of the lateral nasal wall that includes the sphenopalatine artery.

26. The method of claim 24, wherein the step of releasing the compressed device comprises allowing the strut to expand until the pads at opposite ends of the strut bear against opposing wall regions of the body cavity to hold the device in place.

27. The method of claim 24, further comprising performing a surgical procedure at the body cavity while the tamponade device is in place, leaving the tamponade device in place for a predetermined time period after surgery, and removing the tamponade device from the body cavity.

28. The method of claim 24, wherein the device is held in the bent, generally U-shaped configuration in an insertion sheath while the device and sheath are inserted into the body cavity, and the step of releasing the device comprises pushing the device out of the sheath into the predetermined location in the body cavity.

29. The method of claim 28, wherein the step of pushing the tamponade device out of the sheath is carried out by a reciprocating pusher in the sheath which is movable between a retracted position in the sheath and an extended position projecting out of the sheath.

30. The method of claim 29, further comprising holding a bent portion of the elongate strut with a hook at the end of the pusher until the tamponade device completely exits the sheath, and releasing the elongate member from the hook and retracting the pusher into the sheath when the tamponade device is in position.

31. An insertion and retrieval instrument for inserting an arterial tamponade device comprising first and second pads at opposite ends of a flexible, deformable connecting strut into a body cavity to apply pressure to a blood vessel in a cavity wall with the first pad while the second pad bears against an opposing portion of the cavity, comprising:
an outer tubular sheath having a proximal end, a distal end, and a first handle loop closer to the distal end of the sheath;
an elongate pusher member having a distal end portion slidably engaged in the sheath and a proximal end portion extending rearwards out of the proximal end of the sheath, the pusher member having a hook at the distal end configured to engage the bent end of the connecting strut when the strut is bent into a compressed, generally U-shaped configuration, and a second handle loop on the proximal end portion of the pusher member outside the tubular sheath;
the pusher member being movable back and forth in the tubular sheath between a retracted position in which the hook is located within the sheath and an extended position projecting out of the distal end of the sheath;
the first and second handle loops extending parallel to one another in the same direction from the respective tubular sheath and pusher member; and
a biasing device between the first handle loop and tubular sheath configured to bias the pusher member into a retracted position in which the hook is located within the tubular sheath, whereby at least the compressed, U-shaped strut of a tamponade device engaged by the hook is held within the sheath;
whereby a user can extend move the hook out of the sheath by urging the second handle loop towards the first handle loop.

32. The insertion and retrieval instrument of claim 31, further comprising a rearwardly projecting portion at the distal end of the tubular sheath which extends rearwards parallel to the pusher member and spaced from the pusher member, and rear tail fin at the proximal end of the projecting portion, the rear tail fin being configured for engagement by a user's thumb while pulling back on the second handle loop to retract the pusher member into the tubular sheath.
